(19) United States
(12) Patent Application Publication
(10) Pub. No.: US 2012/0203154 A1
(43) Pub. Date: Aug. 9, 2012

(54) Rollable Compact Transparent Splint

(75) Inventor: Barak Tzachar, Petach Tikva (IL)

(73) Assignee: WAISMED LTD., Herzliya (IL)

(21) Appl. No.: 13/355,656

(22) Filed: Jan. 23, 2012

(30) Foreign Application Priority Data
Feb. 3, 2011 (IL) 211046

(51) Int. Cl. A61F 5/00

(52) U.S. Cl. 602/6

(57) ABSTRACT

The invention relates to a rollable splint set which comprises an essentially rectangular substrate, said substrate is made of a transparent or translucent thin layer of thermoplastic material having transverse and longitudinal flexibilities that enable the substrate to assume the following three states: (a) a packed-state in which the splint is maintained in a compact rolled configuration; (b) a pre-use, unrolled state in which the splint automatically assumes a C-configuration in cross section; and (c) a use-state in which the splint wraps an extremity in a C-configuration or a U-configuration in cross section.

### FIELD OF THE INVENTION

The invention relates in general to the field of medical devices. More particularly, the invention relates to a light-weight fixation splint, which is rollable to assume a compact size when packed, and is transparent for enabling visual inspection of the location of injury during use.

### BACKGROUND OF THE INVENTION

Fractures of extremities, such as arms, legs, or fingers are very common. The recovery period of a fractured extremity is relatively long, lasting from several weeks to several months. Fractured injuries have to be treated with care, as mobility of a fractured bone is very painful, can increase the damage, and can extend the recovery period. Therefore, an immediate fixation of an injured extremity, shortly after the event of injury is very preferable. As a result, emergency personnel typically apply an on-site fixation to the injured extremity when they suspect a fracture. More specifically, a splint is applied in the site to the injury until the patient arrives to a doctor for in-depth examination.

Prior art splints are commonly provided as a one-piece elongated rigid device, which is affixed to the injured extremity, for example, by means of tapes or straps. In such a manner, the splint ensures immobility of the bones until arrival the doctor for further examination. Typically, if the doctor's examination shows that the injury is indeed a fracture, a permanent cast is provided for a relatively long duration until full recovery.

Prior art splints suffer from several drawbacks, as follows:
a. Some of them are large in size: As mentioned, typical prior art splints are made of one elongated piece of rigid material. Therefore, they occupy a relatively large space, even when they are not in use.
b. Some of them are relatively cumbersome and heavy: Prior art splints are relatively bulky and heavy, therefore they are inconvenient to the medical personnel and the patient.
c. They are opaque: When a prior art splint is applied, a visual inspection of the injured extremity requires removal of the splint, as the location of injury cannot be inspected via the opaque splint.
d. They do not provide aeration to the injured extremity: Typically, prior art splints comprise a continuous surface, therefore they do not provide aeration to the injured extremity.
e. Each splint has its own shape and length: Each one-piece splint of the prior art has its own shape, and typically cannot be applied to different extremities, namely, a splint for the hand is not adequate to the leg, etc.
f. Some of them are relatively expensive: Therefore, they are typically designed for multiple uses, and are not disposable.

Sam Splint (http://www.sammedical.com/sam_splint.html) discloses a rollable splint which is made of a thin and flat aluminum layer. The Sam splint, when is unrolled, is not ready for use, as it assumes a flat, relatively rigid configuration, and requires performance of a special folding procedure in order to bring it to one of three configurations (in cross section): a C-configuration, a T-configuration, or a reverse C-configuration. The Sam Splint is opaque, and visual inspection of the extremity requires its prior-removal. Moreover, the Sam splint does not provide a manner by which an L-shape fixation can be provided.

It is therefore an object of the present invention to provide a splint which is very compact in size, when packed in its non-use state.

It is another object of the present invention to provide a splint which is very thin, and of light weight, therefore more convenient to the medical personnel and the patient.

It is still another object of the invention to provide a splint which is transparent or translucent, and which therefore enables inspection of the injured extremity also while being in use.

It is still another object of the invention to provide a splint having perforations for aeration to the injured extremity.

It is still another object of the invention to provide a splint which can be directly applied to the skin with no danger of sticking, and which can be easily cleaned for reuse.

It is still another object of the invention to provide a splint which enables the extremity to be X-Rayed, or MRI scanned without removal of the splint.

It is still another object of the present invention to provide a splint which can be adapted on-site to various types, shapes and lengths of extremities.

It is still another object of the present invention to provide a low cost splint, which can be treated as a disposable splint.

Other objects and advantages of the present invention will become apparent as the description proceeds.

### SUMMARY OF THE INVENTION

The invention relates to a thin layer rollable splint having three states: (a) a packed-state in which the splint is maintained in a compact rolled configuration; (b) a pre-use, unrolled state in which the splint automatically assumes a C-configuration in cross section; and (c) a use-state in which the splint wraps the extremity in a C-configuration or a U-configuration in cross section.

Preferably, the splint is maintained in the rolled packed configuration by means of a string.

Preferably, the splint is maintained during said use-state by means of straps.

Preferably, said straps are built-in straps or independent straps. has a thickness of between 0.5 mm and 1.5 mm.

Preferably, the splint is transparent or translucent.

Preferably, the splint further comprises aeration holes.

Preferably, said string is also used to maintain the splint in an L-shape configuration during said use-state, while using two opposing tongues in the splint as connection points.

Preferably, the splint is maintained in a rolled configuration within a tubular device during its packed-state.

Preferably, the splint further comprises notches and marks for cutting to a desired size.

Preferably, the splint is made of thermoplastic polymers.

Preferably, the splint is made of a rigid, elastic, laminar material.

The invention also relates to a splint kit which comprises: (a) a splint having three states as described above; (b) a string for maintaining the splint in a rolled configuration during a packed-state; and (c) straps for affixing the splint to an extremity during a use-state.

Preferably, the kit further comprises scissors for cutting the splint to a desired size.

### BRIEF DESCRIPTION OF THE DRAWINGS

In the drawings:

FIG. 1a shows the fixation splint 1 of the present invention in its pre-use state, according to a first embodiment of the present invention;

FIG. 1b shows an embodiment of the splint of the invention, which is provided with built in straps for affixing the splint to the extremity;

FIGS. 2a-2f show various applications of the splint of the present invention;

FIG. 3 shows the splint of the invention in its use-state, where the splint is affixed to the extremity by means of built-in or independent straps;

FIG. 4 shows the splint in its packed-state, according to a first embodiment of the invention; and

FIGS. 5a and 5b show perspective and upper views respectively of the kit of the invention in its packed-state, according to a preferred embodiment of the invention.

### DETAILED DESCRIPTION OF PREFERRED EMBODIMENTS

The splint of the present invention can assume three states: (a) a packed-state in which the splint assumes a rolled configuration; (b) a pre-use state in which the splint is unrolled, automatically assuming a C-configuration; and (c) a use-state where the splint is applied to the extremity in a C-configuration in cross section, or when a higher rigidity is desired, in a U configuration.

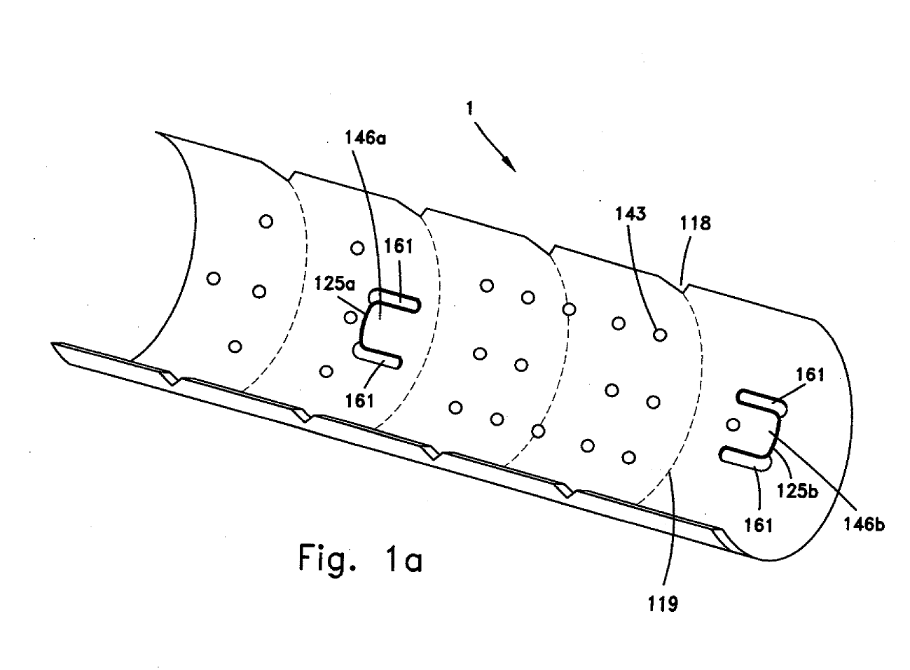

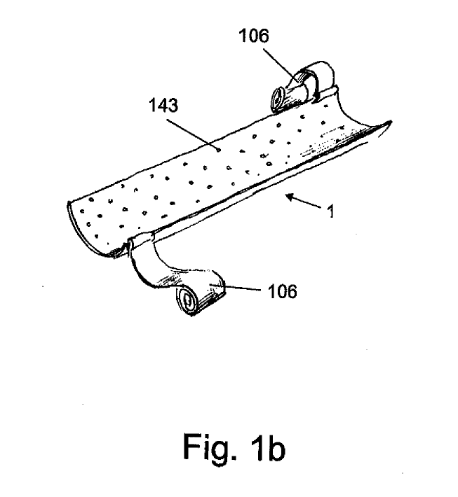

FIG. 1a shows the fixation splint 1 of the present invention in its pre-use state, according to a first embodiment of the present invention. FIG. 1b shows another embodiment of the splint of the invention, which is provided with built in straps for affixing the splint to the extremity. The splint is essentially rectangular and relatively thin sheet of material, having slightly concave cross-section in its pre-use state. The “slightly concave” cross section of the splint in its pre-use state will be referred to herein as “C-configuration”. As will be further demonstrated hereinafter, the radius of curvature of the cross section of the splint in its C-configuration is larger compare to the respective radius of curvature of the splint in its “U-configuration”. The rectangular shape of the substrate defines two directions, longitudinal and transverse respectively.

The splint is preferably made of thermoplastic polymers, such as polycarbonate, polypropylene, polystyrene, etc. The thermoplastic polymers splint is preferably prepared by a heat-pressing process.

The substrate is transparent or translucent to enable visual inspection of the injury through the splint. In one preferred embodiment, the splint is made of transparent, flexible polycarbonate. The thickness of the splint may be in the range of 0.5 mm to 1.5 mm, for example, 0.75 mm, and may have various dimensions, for example, 70×12 cm. The splint is flexible about longitudinal and transverse axes; however, as will be demonstrated hereinafter, the flexibility of the splint about a transverse axis is significantly lower in its U-configuration compared to the respective flexibility in its C-configuration. Therefore, the optional U-configuration is used when higher rigidity is needed.

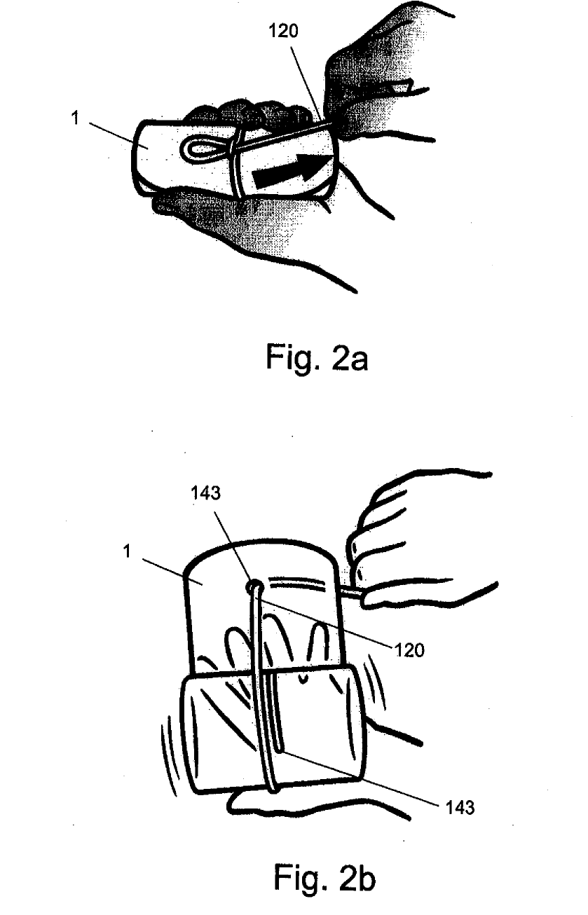

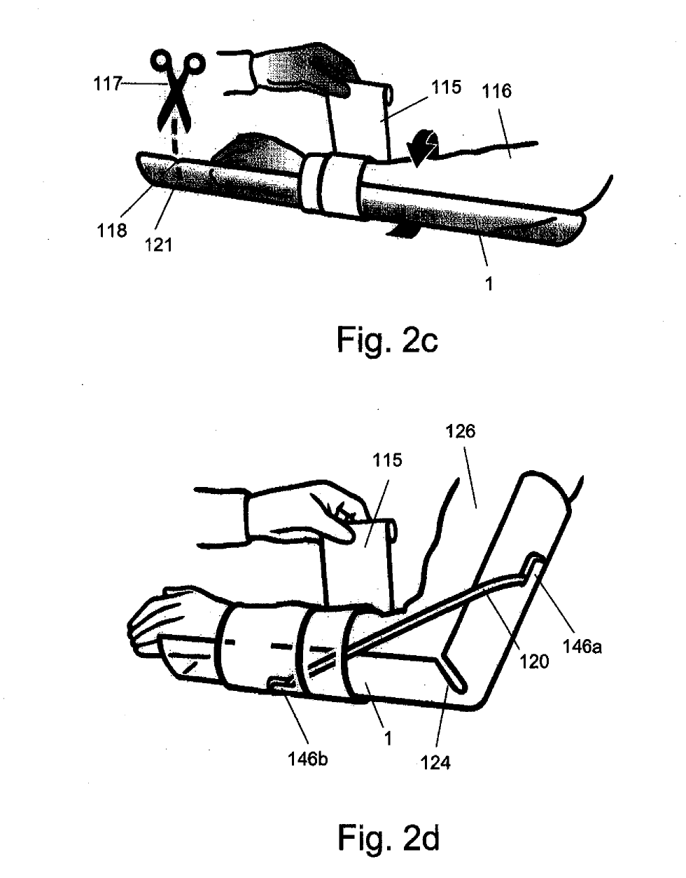

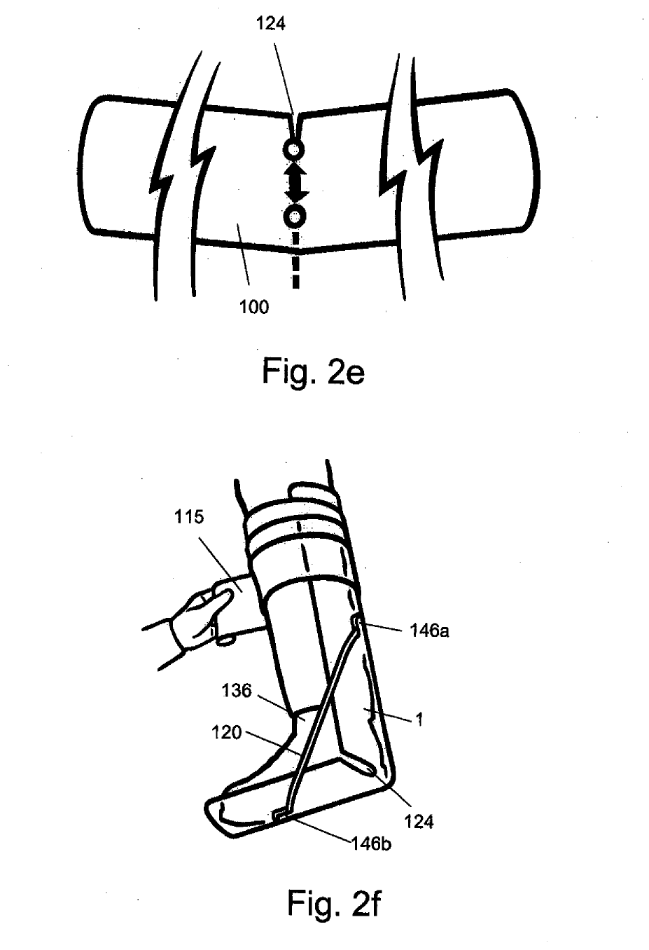

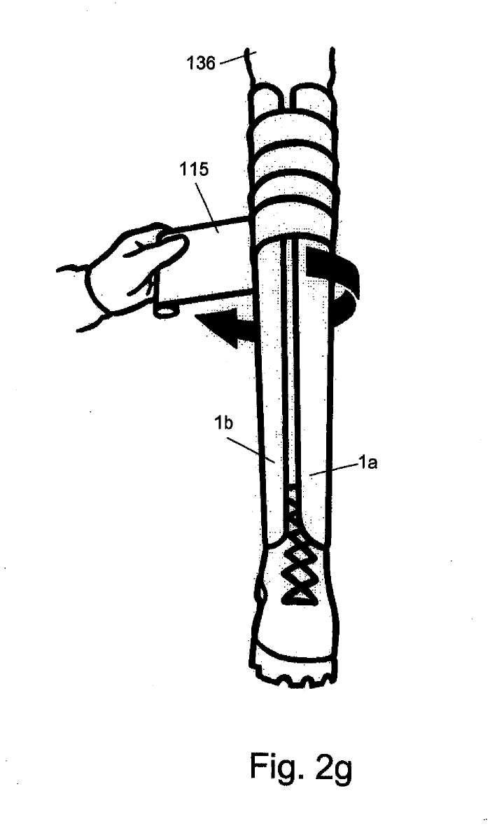

The splint may include perforations 143 (hereinafter, “aeration holes”) for aerating the extremity during the in-use state. The low thickness of the splint enables it to be cut on-site to the desired length, thereby adapting it to various types and lengths of extremities, and also to various situations. For example, when an L-shape fixation is necessary, partial transverse cuts 124 (shown in FIG. 2f) from the two sides may be made to enable folding of the splint about a transverse axis to form an L-shaped splint. Several openings within the substrate may serve still other purposes. For example, two opposite U-shaped cuts 125a and 125b at the two opposite ends of the splint may be provided for maintaining the splint by means of a string in an L-shape configuration during use, if so desired. In that case, each of the two respective U-shaped cuts 125a and 125b forms a tongue 146a and 146b, which is used as a hook for a fastening string 120 as shown in FIG. 2f. Adjacent the U-shaped cuts 125a and 125b, additional openings may be provided for the sake of convenience. The aeration holes 143 may also be used for the insertion of string 120, which keeps the splint in a rolled configuration during its packed-state as shown in FIG. 2a.

As noted above, the splint of the present invention can be rolled to assume a compact size in its packed-state. In a first embodiment shown in FIGS. 4a and 4b, the splint 1 is rolled, while string 120 passes through aeration holes 143 in all the layers that are formed in the rolled configuration, to ensure maintaining it in this rolled state, and additionally, to provide a braking action on the splint's tendency to unroll itself when it is released from its packed configuration. In still another embodiment shown in FIG. 4, in its packed-state the rolled splint 1 is contained within the tube 210. In this embodiment, the unpacking of the splint 1 is performed through slot 214.

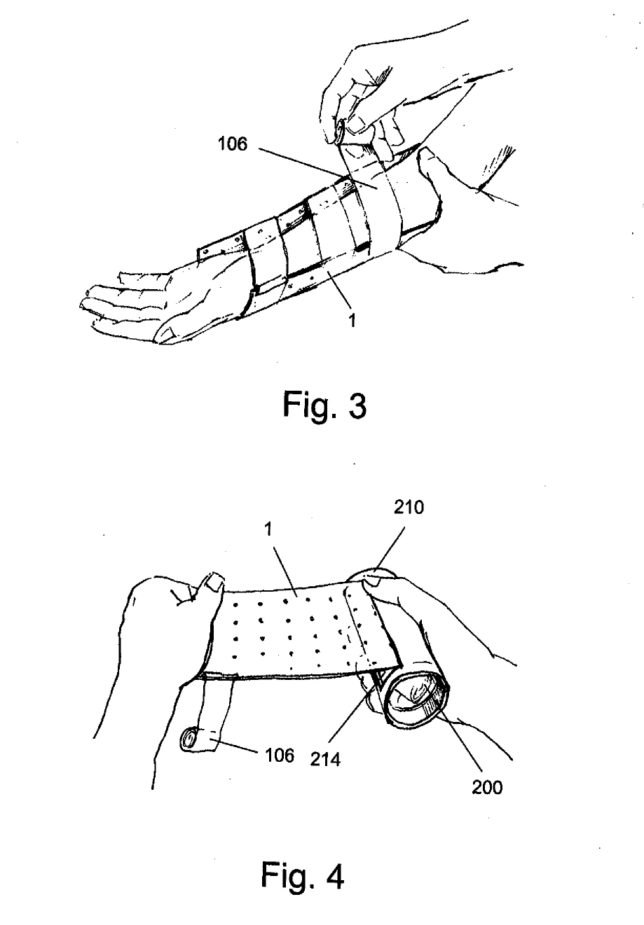

FIG. 3 shows the splint 1 in its use-state, where the splint is affixed to the extremity by means of built-in or independent straps 106. In the use-state the splint may be in either the C-configuration or pressed to a U-configuration in cross section when higher rigidity is necessary. In its U-configuration, the rigidity of the splint 1 to fold about a transverse axis is significantly increased compared to its rigidity in the C-configuration of FIGS. 1a and 1b (where the radius of curvature of the C-configuration is much larger). As noted, on one hand the splint can be rolled to a very compact size in its packed-state, and on the other hand it can serve the fixation functionality (in the use-state) with a C-configuration of lower rigidity or U-configuration of higher rigidity. Moreover, it can be used in various types of extremities and situations.

FIGS. 4a-4g demonstrate various manners of use of the splint of the present invention. FIG. 2a shows the splint 1 in its rolled, packed-state, wherein it is maintained in this state by means of string 120. FIG. 2b shows a next step of unrolling the splint 1. As shown, when rolled in the packed-state, the string 120 passes through a plurality of holes 143 in several layers of the splint that are formed due to the rolling. By this means it becomes possible to gradually release the rolled splint, by gradually releasing the string 120 from its previously tied state, the friction between string and holes providing a braking force which opposes the tendency of the splint to unroll itself. At the end of the unrolling process, the splint 1 is brought to its pre-use state, as shown in FIGS. 1a and 1b. FIG. 2c shows a manner of affixing the splint 1 to the extremity 116 (in this case arm). The arm 116 is brought into the concavity of splint 1, which in this case is pressed to assume a high rigidity U-configuration, and then it is affixed in this U-configuration to the arm by means of (optionally sticky) straps 115. Alternatively, the splint may be used in a similar manner in its C-configuration, when lower rigidity is found to be sufficient. If the splint is found to be too long, it can be shortened by use of scissors 117. Preferably, the splint has several notches 118 (shown in FIG. 1a), and transverse lines 119 at selected locations to enable easy cutting of the splint (although cutting can, in fact, be made at any desired location, even at a location with no notches or transverse line). FIG. 2d shows how the splint can be used for fixation of a whole hand 126. For this purpose, the splint is transversely but partially cut from the two sides as demonstrated in FIG. 2e, forming two notches 124 (only one is shown in FIG. 2e). The notches from the two sides enable easy folding of the splint to an essentially L-shape arrangement, as shown in FIG. 2d. Again, straps 115 affix the splint 1 to the hand. String 120, which was originally used to maintain the splint in its packed-state (see FIGS. 4a and 4b) is now used to firmly maintain the splint in the L-shape configuration. The string 120 is affixed to the splint by means of the two tongues 146a and 146b, each forming a hook-like element, that are provided at two opposite locations of the splint. FIG. 2f shows fixation of a leg 136 by means of the splint 1, which is performed in a similar manner as described with respect to FIGS. 4d and 4e. FIG. 2g demonstrates fixation of a leg by means of two splints 1a and 1b.

The invention also relates to a splint kit, which comprises:
a. A rollable splint as described above;
b. A string for maintaining the splint in a rolled configuration during a packed-state, or for maintaining it in an L-shape, when desired, during a use-state; and
c. Straps for affixing the splint to the injured extremity, said straps may also be provided in a rolled configuration.

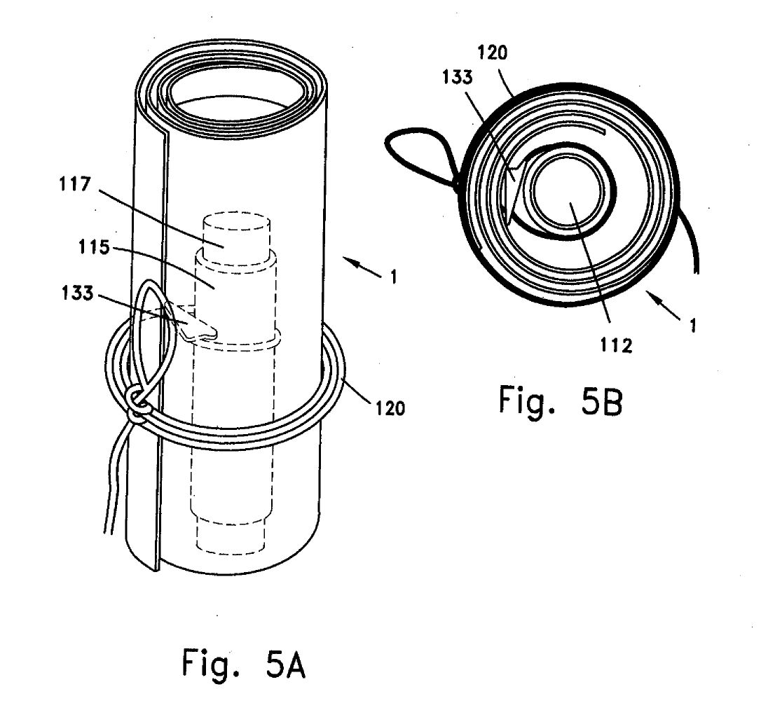

FIGS. 5a and 5b show perspective and upper views respectively of the kit of the invention in its packed-state. The packed splint 1 is shown in its rolled configuration, forming a generally tubular shape. The splint is maintained in said rolled state by means of string 120. The straps 115, that are also provided in a rolled configuration over a cylindrical core 112 are maintained within the tube by means of string 120. The string is fitted with a cleat or toggle 133, which is used in various applications of the splint, to maintain the string at a desired length and tension. As shown, the kit of the splint, which comprises the splint 1 in its packed configuration, the straps 115, and the string 120, is very compact in size.

Optionally, the kit may also comprise Scissors (not shown) for optional cutting of the splint 1 to a desired size, or for partial cutting to enable fold of the splint at a desired location.

While some embodiments of the invention have been described by way of illustration, it will be apparent that the invention can be carried out with many modifications, variations and adaptations, and with the use of numerous equivalents or alternative solutions that are within the scope of persons skilled in the art, without departing from the spirit of the invention or exceeding the scope of the claims.

1. A rollable splint set which comprises an essentially rectangular substrate which is made of a transparent or translucent thin layer of thermoplastic material having transverse and longitudinal flexibilities that enable the substrate to assume the following three states:
a. a packed-state in which the splint is maintained in a compact rolled configuration;
b. a pre-use, unrolled state in which the splint automatically assumes a C-configuration in cross section; and
c. a use-state in which the splint wraps an extremity in a C-configuration or a U-configuration in cross section.

2. A splint set according to claim 1, further comprising a string for maintaining the substrate in said rolled packed configuration.

3. A splint set according to claim 1 further comprising plurality of straps for maintaining the splint on the extremity during said use-state.

4. A splint set according to claim 3, wherein said straps are built-in straps or independent straps.

5. A splint according set to claim 1, wherein the rectangular substrate has a thickness of between 0.5 mm and 1.5 mm.

7. A splint set according to claim 1, wherein the substrate further comprises aeration holes.

8. A splint set according to claim 2, wherein said string is also used to maintain the splint in an L-shape configuration during said use-state, while using two opposing tongues in the splint as connection points.

9. A splint set according to claim 1, further comprising a tubular device for maintaining the substrate in a rolled configuration during its packed-state.

10. A splint set according to claim 1, wherein the substrate further comprises notches and marks for ease cutting of the substrate to a desired size.

11. A splint set according to claim 1, wherein said substrate is transparent to CT, X-Ray, or both.

12. A splint set according to claim 1, wherein the substrate is made of thermoplastic polymers.

13. A splint set according to claim 1, wherein the substrate is made of a rigid, elastic, laminar material.

14. A splint kit which comprises:
a. a splint set according to claim 1;
b. a string for maintaining the splint in a rolled configuration during a packed-state; and
c. straps for affixing the splint to an extremity during a use-state.

15. A splint kit according to claim 13, further comprising scissors for cutting the splint to a desired to size.
